(19) United States
(12) Patent Application Publication
(10) Pub. No.: US 2004/0138200 A1
(43) Pub. Date: Jul. 15, 2004

(54) Pharmaceutical compositions for treatment of Parkinson's disease

(76) Inventors: Michael Hawley, Kalamazoo, MI (US); Michael S. Bergren, Portage, MI (US); Phillip R. Nixon, Portage, MI (US); Gordon W. Halstead, Scotts, MI (US); Robert S. Chao, Santa Clara, CA (US); Tzu-chi R. Ju, Vernun Hills, IL (US)

Correspondence Address: Saul L. Zackson, Harness, Dickey & Pierce, P.L.C., Suite 400, 7700 Bonhomme Avenue, St. Louis, MO, 63105, US

(21) Appl. No.: 10/677,778

(22) Filed: Oct. 2, 2003

Related U.S. Application Data
(60) Provisional application No. 60/416,296, filed on Oct. 4, 2002.

(51) Int. Cl. A61K 31/553; A61K 31/554; A61K 31/551; A61K 31/542; A61K 31/538

(52) U.S. Cl. 514/211.12; 514/220; 514/224.5; 514/229.8; 514/291; 514/250

(57) ABSTRACT

A pharmaceutical composition comprising a salt, preferably a crystalline salt, of an organic acid of low water solubility and a compound of formula (I) is provided. The salts are useful in treating Parkinson's disease. Also provided are methods of treatment using the salts and methods of making crystalline forms of the salts.

This application claims the benefit of U.S. Provisional Application No. 60/416,296, filed Oct. 4, 2002.

### BACKGROUND OF THE INVENTION

(1) Field of the Invention

The present invention relates to tricyclic, nitrogen-containing compounds, which are heterocyclic amines, and salts thereof, and, in particular to salts of low water solubility of the compounds. The salts are suitable for use in extended release formulations for treating Parkinson's disease.

(2) Description of the Related Art

Tricyclic, nitrogen-containing compounds, which are therapeutically useful in treating neurological diseases such as Parkinson's disease are independently disclosed in the patents and publications individually cited immediately below.

U.S. Pat. No. 5,273,975 to Moon et al.

U.S. Pat. No. 6,197,339 to Ju.

International Publication WO 99/16442 to Ju.

International Publication WO 00/40226 to Meglasson et al.

The disclosed compounds have the structure of formula (I) as described hereinbelow. Methods of synthesis, properties and uses of compounds of formula (I) are disclosed in these references, as are pharmaceutically acceptable salts of the compounds. The disclosed salts are salts of acids which have a high water solubility. For example, the acids of the salts disclosed in International Application WO 00/40226 are preferably selected from methanesulfonic acid, hydrochloric acid, hydrobromic acid, sulfuric acid, phosphoric acid, nitric acid, benzoic acid, citric acid, tartaric acid, fumaric acid, maleic acid, CH₃—(CH₂)ₙ—COOH, where n is 0 thru 4, and HOOC—(CH₂)ₙ—COOH, where n is defined as above. Similarly, U.S. Pat. No. 5,273,975 discloses salts of acids which are preferably selected from hydrochloric, hydrobromic, hydroiodic, sulfuric, phosphoric, acetic, propionic, lactic, maleic, malic, succinic, tartaric, cyclohexanesulfamic, methanesulfonic, ethanesulfonic, benzenesulfonic, toluenesulfonic and other pharmaceutically acceptable acids of counter ions for amines. International Publication WO 99/16442 and U.S. Pat. No. 6,197,339 describe a maleate salt of (R)-5,6-dihydro-5-(methylamino)-4H-imidazo[4,5,1-ij]quinolin-2(1H)-one, a compound of formula (I). None of these references disclose salts of acids of low water solubility.

### BRIEF DESCRIPTION OF THE INVENTION

In accordance with the present invention, the inventors herein have succeeded in discovering salts of compounds of formula (I) which are prepared from acids of low water solubility. The salts are preferably substantially crystalline in form. The salts can be used in extended release formulations which are particularly useful for treatment of neurological disease such as Parkinson's disease.

Thus, in one embodiment, the invention comprises a pharmaceutical composition comprising a salt, the salt comprising a compound of formula (I)

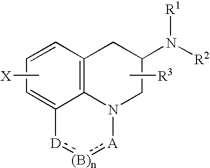

where R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;

X is: —H, C₁-C₆ alkyl, —F, —Cl, —Br, —I, —OH, C₁-C₆ alkoxy, cyano, carboxamide, carboxyl, (C₁-C₆ alkoxy)carbonyl;

A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;

B is: CH₂, CH, CH-(halogen) where halogen is as defined above, C═O, N, NH, N—CH₃,

D is: CH, CH₂, CH-(halogen) where halogen is as defined above, C═O, O, N, NH, N—CH₃; and n is 0 or 1, and where [symbol] is a single or double bond, with the provisos:

(1) that when n is 0, and

A is CH₂, CH-(halogen) where halogen is as defined above, CHCH₃, C═O, C═S, C═NH, SO₂; then

D is CH₂, CH-(halogen) where halogen is as defined above, C═O, O, NH, N—CH₃;

(2) that when n is 0, and

A is CH, C—SCH₃, C—NH₂, C—NHCH₃, C—NHCOOH₃, C—NHCN, N; then D is CH, N;

(3) that when n is 1, and

A is CH₂, CH-(halogen) where halogen is as defined above, CHCH₃, C═O, C—S, C═NH, SO₂; and

B is CH₂, CH-(halogen) where halogen is as defined above, C═O, NH, N—CH₃; then

D is CH₂, C═O, O, NH, N—CH₃;

(4) that when n is 1, and

A is CH, C—SCH₃, C—NH₂, C—NHCH₃, C—NHCOOCH₃, C—NHCN, N; and

B is CH, N; then

D is CH₂, C═O, O, NH, N—CH₃;

(5) that when n is 1, and

A is CH₂, CHCH₃, C═O, C═S, C—NH, SO₂, and

B is CH, N; then

D is CH, N.

The salt further comprises an organic acid having low water solubility. An organic acid having low water solubility has a water solubility of less than about 1% (w/w), preferably, less than about 0.1% (w/w), more preferably, less than about 0.01% (w/w). Preferred organic acids are naphthylenecarboxylic acids and derivatives thereof having the structure

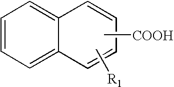

where R₁ is selected from the group consisting of hydroxy, amino, and nitro (formula (A));

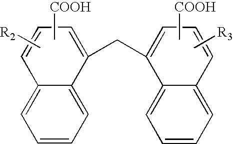

where R₂ and R₃ are independently selected from the group consisting of hydroxy, amino, and nitro (formula (B)).

A preferred acid of formula (B) is pamoic acid (4,4′-methylenebis[3-hydroxy-2-napthalenecarboxyolic acid]) and a preferred acid of formula (A) is xinafoic acid (1-napthol-2-carboxylic acid).

In another aspect, other acids of low water solubility can be used, including long-chain carboxylic acids having the structure, CH₃(CH₂)ₙCOOH wherein n is an integer from 5 to 16 or greater (formula (C)). Preferably, a long-chain carboxylic acid of formula (C) has the structure, CH₃(CH₂)ₙCOOH wherein n is an integer from 5 to 16 “Long-chain carboxylic acids having the structure, CH₃(CH₂)ₙCOOH wherein n is an integer from 5 to 16” are linear carboxylic acids and isomers thereof, for example branched chain carboxylic acids and cyclic carboxylic acids, comprising from 7 to 18 carbon atoms. Additional acids of low water solubility can also be used, such as long-chain dicarboxylic acids having the structure, HOOC(CH₂)ₘCOOH wherein m is an integer from 5 to 16 or greater (formula (D)). “Long-chain dicarboxylic acids having the structure, HOOC(CH₂)ₘCOOH wherein m is an integer from 5 to 16” are linear dicarboxylic acids and isomers thereof, for example branched chain dicarboxylic acids and cyclic dicarboxylic acids, comprising from 7 to 18 carbon atoms.

In one aspect, the salts of the present invention are substantially crystalline. A crystalline salt of the present invention has a rate of dissolution in a water medium slower than that of a maleate crystalline salt of the compound. In preferred embodiments, a salt of the invention has a dissolution rate into aqueous solution of about 0.40 mmole sec⁻¹ cm⁻² or less under standard dissolution conditions. More preferably, the salt of the invention has a dissolution rate into aqueous solution of about 0.20 mmole sec⁻¹ cm⁻² or less under standard dissolution conditions. More preferably, the salt of the invention has a dissolution rate into aqueous solution of about 0.05 mmole sec^{31 1} cm⁻² or less under standard dissolution conditions.

The salts and in particular, the crystalline salts of the present invention are substantially insoluble in water and for this reason, the material can be incorporated into formulations which exhibit extended or sustained release characteristics. Thus, in one embodiment, the invention is directed to an extended release dosage formulation comprising a therapeutically effective amount of one of the salts of the present invention which is preferably in a crystalline form.

A therapeutically effective amount of the pharmaceutical compound is provided as the salt dissolves in the gastrointestinal tract of the subject over a time course of, preferably, at least about 12 hours, and more preferably, about 24 hours. The extended release formulation is thus suitable for administering to a patient at a frequency of, preferably, not more frequently than twice a day and more preferably, not more frequently than once a day.

In another aspect, the present invention includes methods of making a crystalline form of a pamoic acid salt a compound of formula (I) or a crystalline form of a xinafoic acid salt of a compound of formula (I). The method comprises dissolving both the compound and the acid in a first solvent, removing at least a portion of the first solvent, redissolving in a second solvent, and crystallizing the salt from the second solvent. The first solvent is preferably methanol or pyridine, and the second solvent is preferably acetonitrile or a mixture of methanol and acetonitrile.

The present invention also includes methods of treating a neurological disease, in particular, Parkinson's disease in a subject. The method comprises orally administering to the subject, a composition comprising a salt of the present invention or an extended release formulation comprising a salt of the present invention.

### BRIEF DESCRIPTION OF THE DRAWING

FIG. 1 illustrates rotating disc dissolution data for maleate and xinafoate salts of (R)-5,6-dihydro-5-(methylamino)-4H-imidazo[4,5-ij]-quinolin-2(1H)-one (sumanirole).

### DETAILED DESCRIPTION OF THE INVENTION

The present invention provides pharmaceutical compositions comprising a salt, the salt comprising a compound of formula (I) and an organic acid having low water solubility. The organic acid can be a naphthylenecarboxylic acid or derivative thereof of formula (A) or formula (B), preferably pamoic acid or xinafoic acid, a long chain carboxylic acid of formula (C), in which n is an integer preferably of 5-16, or a long chain dicarboxylic acid of formula (D), in which m is an integer preferably of 5-16. The salts are useful in extended release compositions for treating neurological diseases such as Parkinson's disease in a subject. By treatment as referenced herein, it is meant that a pharmaceutical composition is given to a subject either prophylactically or therapeutically.

Administration of the compositions of the present invention preferably comprises administration of the compositions to a human subject, although administration within the present invention can also comprise administration to a non-human mammal. A non-human mammal can be, for example, a commercial animal, (including non-limiting examples such as horses, cattle, swine, sheep and transgenic mice) and exotic animal such as zoo animals, sporting animals (including non-limiting examples such as horses and dogs) as well as companion animals (including non-limiting examples such as dogs and cats).

The organic acid component of the salt of the present invention, has a low water solubility. Solubility as referenced herein is intended to reference the amount of a substance which dissolves or forms a solution in distilled water at 25° C. Unless otherwise indicated, solubility is represented as percent weight solute/weight solvent (w/w), i.e., the amount of a substance in grams dissolved in 100 grams of water.

Preferred compounds of formula (I) are sumanirole, i.e., (R)-5,6-dihydro-5-(methylamino)-4H-imidazo[4,5,1-ij] -quinolin-2(1H)-one, (R)-5,6-dihydro-5-(methylamino)-4H-imidazo[4,5-ij]-quinoline-2(1H)-thione and (R)-5,6-dihydro-N,N-dimethyl-4H-imidazo[4,5,1-ij]quinolin-5-amine.

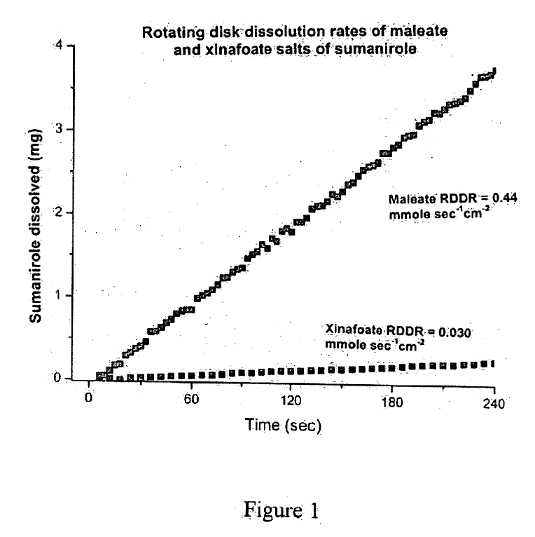

In a preferred aspect, the salts of the present invention are crystalline salts. A crystalline salt of the invention dissolves slower than the crystalline maleate salt of the same compound under standard conditions of temperature, pressure, and fluid flow. Preferably, the dissolution rate of a crystalline salt of the invention, when measured by a rotating disk dissolution test, is less than 50% of that of a crystalline maleate salt of the same compound. More preferably, the dissolution rate is less than 25% of that of a crystalline maleate salt of the same compound. More preferably, the dissolution rate is less than 10% of that of a crystalline maleate salt of the same compound. For example, under the standard conditions in a rotating disc dissolution test, the maleate salt crystals dissolved at a rotating disc dissolution rate (RDDR) of 0.44 mmole sec⁻¹ cm⁻², whereas the xinafoate salt crystals dissolved at a rate of 0.03 mmole sec⁻¹ cm⁻² (see FIG. 1). In preferred embodiments, the salt of the invention the salt has a dissolution rate into aqueous solution of about 0.40 mmole sec⁻¹ cm⁻² or less. In more preferred embodiments, the salt of the invention the salt has a dissolution rate into aqueous solution of about 0.20 mmole sec⁻¹ cm⁻² or less. In more preferred embodiments, the salt of the invention the salt has a dissolution rate into aqueous solution of about 0.05 mmole sec⁻¹ cm⁻² or less.

In certain aspects, a formulation of the present invention can comprise a salt, preferably a crystalline salt, and a pharmaceutically acceptable polymer such as a protein, a starch, a cellulose, or a synthetic polymer. Polymers known in the art, for example those independently disclosed in patents and publications individually cited immediately below, are used to modulate the rate of release of the compound into the aqueous phase.

U.S. Pat. No. 4,389,393.

U.S. Pat. No. 5,000,962.

U.S. Pat. No. 5,614,218.

U.S. Pat. No. 5,658,919.

U.S. Pat. No. 5,922,342.

U.S. Pat. No. 6,197,339.

U.S. Pat. No. 6,322,813.

International Publication WO97/34932.

International Publication WO97/37639.

The polymer is preferably a naturally occurring polymer or a chemically modified derivative thereof, such as a starch (for example, corn starch, wheat starch, pregelatinized starch, or a sodium starch glycolate), a protein (for example, bovine, porcine, or ovine gelatin); or a cellulose (for example, hydroxypropyl methylcellulose, a cellulose ether, or cellulose acetate) and/or a synthetic polymer (for example, a polyvinyl chloride, a polyvinyl acetate, a polyurethane, or a polyalkyl methacrylate). When a starch is included as a matrix, it is preferred that the starch be corn starch, pregelatinized starch, or a mixture thereof. When hydroxypropyl methylcellulose is included in a formulation, the hydroxypropyl methylcellulose is any pharmaceutically acceptable hydroxypropyl methylcellulose, individually or mixtures thereof. It is preferred that when hydroxypropyl methylcellulose is included in the formulation, that it is selected from the group consisting of hydroxypropyl methylcellulose 2208 USP 100 cps, hydroxypropyl methylcellulose 2208 USP 4,000 cps, hydroxypropyl methylcellulose 2208 USP 15,000 cps, hydroxypropyl methylcellulose 2208 USP 100,000 cps, hydroxypropyl methylcellulose 2910 USP 4,000 cps, hydroxypropyl methylcellulose 2910 USP 10,000 cps, or mixtures thereof. It is more preferred that the hydroxypropyl methylcellulose is selected from the group consisting of hydroxypropyl methylcellulose 2208 USP 4,000 cps and hydroxypropyl methylcellulose 2910 USP 4,000 cps. In addition, tablets or capsules can also comprise other agents or inert ingredients known to those of skill in the art, such as coloring agents, fillers, and coatings.

Pharmaceutically acceptable water-insoluble lubricants can be present in an extended-release formulation of the invention. Non-limiting examples of lubricants include glyceryl behapate, stearates (e.g., magnesium, calcium or sodium stearates), stearic acid, hydrogenated vegetable oils, colloidal silica, talc, waxes and mixtures thereof. One or more water-insoluble lubricants can be present in a formulation of the invention in a total amount of about 0.05% to about 5% by weight of the formulation. Preferably, the lubricant is magnesium stearate. If magnesium stearate is present, it can be in an amount of from about 0.2% to about 2.0%.

Pharmaceutically acceptable coatings can be present in a formulation of the invention, for example to enhance flow of tableting material into tablet dies, to prevent sticking of tableting material to punches and dies, or to produce tablets having a sheen. Acceptable coatings include, for example, colloidal silicon dioxide products such as fumed silica (such as Cab-O-Sil™ of Cabot Corp. or Aerosil™ of Degussa). If colloidal silicon dioxide is present, it is preferably in an amount from about 0.2% to about 1.0% (w/w). Other ingredients that are conventional in tablet making that can be added, for example, talc, titanium dioxide, magnesium salts, calcium salts, aluminum salts, lactose, povidone, high molecular weight polyethylene glycols and derivatives, bioerodible polymers such as poly(orthoesters) and polyanhydrides and anhydride co-polymers, polyoxystearates, carboxymethyl cellulose, cellulose ethers such as acetate phthalate, acetate succinate, and cellulose acetate N,N-diethylamino acetate, and polyvinyl alcohol.

Pharmaceutically acceptable sweeteners can be present in a tablet of the invention in a sweetening effective amount. Sweeteners include, for example, sucrose, mannitol, propylene glycol, sodium saccharin, acesulfame K, neotame and aspartame. Furthermore, pharmaceutically acceptable flavoring agents can also be present in a tablet of the invention in a flavoring effective amount. Flavoring agents include, for example, peppermint, spearmint, grape, cherry, strawberry, and lemon.

Tablets of the invention are made to any suitable size, for example 10 mm in diameter; shape, for example round, oval, or oblong; weight; and thickness. Tablets of the invention can also have etchings or monograms on one or both sides. Tablets can also be scored for convenient breaking into smaller segments.

Tablets of the invention have a hardness that depends on size and shape as well as on composition, among other characteristics. Tablet hardness is measured by any method known in the art, for example by a tablet hardness meter. Preferably, compositions of the invention have a hardness of about 1 to about 10 kg, and more preferably of about 1 to about 5 kg.

In a preferred formulation, solid dosage forms of the invention have sufficient hardness for handling. The term “sufficient hardness for handling” as used herein means a hardness that withstands removal from at least a 'standard type of blister packaging, or other handling, for example packaging, delivery, or carrying. A suitable hardness is about 1 kg or more for a tablet having a diameter of about 8 mm, about 1.5 kg or more for a tablet having a diameter of about 10 mm, and about 2 kg or more when the tablet has a diameter of about 12 mm. Preferably, the hardness of the tablets is such that a plurality of tablets packaged together, for example in a glass or plastic bottle, do not exhibit substantial breakage, adherence to neighboring tablets or the packaging material, or melding during normal shipping and handling. Tablets intended for such packaging preferably have a hardness of about 3 kg or more.

Tablets of the invention can be packaged in any suitable manner known in the art. A multiplicity of tablets can be packaged together, for example in a glass or plastic bottle or container. Alternatively, tablets of the invention can be individually wrapped, for example in plastic or foil, or packaged in known forms of blister packaging.

In one embodiment, the formulation is a tablet prepared by direct compression, and comprises a crystalline salt of the present invention, a non-direct compression filler, preferably a non-direct compression sugar or sugar alcohol, for example dextrose, mannitol, sorbitol, lactose, or sucrose, and a lubricant, substantially as disclosed in U.S. Pat. No. 6,024,981.

The invention provides in one embodiment methods of making a composition comprising a crystalline salt, the crystalline salt comprising an organic acid having low water solubility and a pharmaceutical compound of formula (I). Preferably, the organic acid has a water solubility of about 0.1% (w/w) or less. The method comprises dissolving both the compound and the organic acid in a first organic solvent, thereby forming a mixture, then removing the solvent to form a crusted solution or an oil. The first solvent is any solvent in which both the organic acid having low water solubility and the pharmaceutical compound have a solubility of at least 1% (w/w), more preferably, at least 3% (w/w). Preferably, the first solvent is an organic base or a lower alcohol comprising 1 to 3 carbon atoms. More preferably, the first solvent is pyridine when the organic acid is pamoic acid, or a lower alcohol comprising 1 to 3 carbon atoms, preferably methanol, when the organic acid is xinafoic acid. Preferably, the organic acid and compound are mixed in about equimolar ratio. “About equimolar ratio” is preferably a molar ratio of about 1:5 to about 5:1, more preferably a molar ratio of about 1:2 to about 2:1 more preferably a molar ratio about 1:1.4 to about 1.4:1. The first solvent is removed preferably by evaporating the solvent, but any suitable method known in the art, for example chromatography or lyophilization, can also be used. The removing preferably removes at least about 50% of the first solvent, more preferably at least 90% of the first solvent, more preferably at least 99% of the first solvent. The residue remaining upon removal of the first solvent is then redissolved in a second solvent. The second solvent is preferably selected from the group consisting of a lower alcohol comprising 1-3 carbon atoms, acetonitrile, and a mixture thereof. Preferably, the lower alcohol is methanol. More preferably, the second solvent is a mixture of methanol and acetonitrile when the organic acid is pamoic acid, or acetonitrile when the organic acid is xinafoic acid. In the former case, the lower alcohol and the acetonitrile are initially added in approximately equal volumes, and gentle heating can be used to promote dissolution: The volume ratio of acetonitrile to lower alcohol is then increased by decreasing the amount of the lower alcohol and/or increasing the amount of the acetonitrile. For both solutions, a crystalline salt comprising the organic acid and the pharmaceutical compound is formed upon removal of the second solvent. Removal of the second solvent from the crystalline salt can be by any method known to persons of skill in the art. Preferably, the second solvent is removed by evaporation of the second solvent and/or by spontaneous precipitation of salt crystals from the second solvent. In the latter case, the second solvent is further removed following crystal formation by standard methods known to persons of skill in the art such as filtration, evaporation, or lyophilization. These procedures yield a crystalline salt comprising an organic acid having low water solubility and the pharmaceutical compound of formula (I). Preferably, the organic acid has a water solubility of about 0.1% (w/w) or less.

Methods of making the salts and, in particular, the crystalline salts of the present invention, as well as some physical properties of the salts, including dissolution properties in an aqueous environment, are illustrated in the examples provided below.

### EXAMPLE 1

This example illustrates the preparation of crystals of the pamoate salt of sumanirole.

Sumanirole, 2.078 grams (10.22 mMole), and pamoic acid, 1.984 grams (5.108 mMole) were added to a 25 ml flask. Nine ml of pyridine was then added, and the mixture was stirred until all the components were dissolved. Forced evaporation of the pyridine overnight yielded a solution with a crust on top. Three ml of methanol and 3 ml of acetonitrile were then added to the mixture, and the solids were redissolved with heating. The mixture was then placed in a rotary evaporator, and the volume was reduced by evaporation to about 5 ml. An additional 10 ml of acetonitrile was then added. This mixture, upon evaporation in a rotary evaporator, produced crystals. About 3 grams of crystalline product was isolated (˜75% yield).

### EXAMPLE 2

This example illustrates the preparation of crystals of the xinafoate salt of sumanirole.

Sumanirole, 103.5 mg (0.509 mMole) and 1-hydroxy-2-napthoic acid, 105 mg (0.558 mMole) were added to a 20 ml vial. Three ml of methanol was added to the solids. Both the acid and the pharmaceutical compound dissolved at ambient temperature. The methanol solvent was allowed to evaporate off under an inverted 250 ml beaker. The evaporation left an oil residue. One ml of acetonitrile was then added to the oil, and the mixture was stirred at ambient temperature. An oily solution resulted. Stirring of the solution was stopped, and the solution was allowed to stand overnight. Crystals precipitated from the solution (172 mg, 86% yield).

### EXAMPLE 3

This example describes the results of analyses of physical characteristics of the crystals obtained in Examples 1 and 2.

Infrared spectroscopic analyses were performed on samples of each salt of Examples 1 and 2 using a scanning infrared spectrophotometer. The resulting spectrograms of IR absorbance were consistent with each structure.

Table 1 presents analytical results for the sumanirole pamoate of Example 1.

TABLE 1

| Assay | Calculated | Result |
|---|---|---|
| Carbon | 68.00 | 68.08 |
| Hydrogen | 5.33 | 5.27 |
| Nitrogen | 10.57 | 10.63 |
| KF water* | — | 0.17% |
| ROI** | — | 0.03% |

*Water content as determined by the Karl Fischer assay.
**Residue on Ignition

Table 2 presents analytical results for the sumanirole xinafoate of Example 2.

TABLE 2

| Assay | Calculated | Result |
|---|---|---|
| Carbon | 67.51 | 67.43 |
| Hydrogen | 5.41 | 5.41 |
| Nitrogen | 10.74 | 10.74 |
| KF water* | — | 0.11% |
| Equivalent weight | 380-403 | 393 |

*Water content as determined by the Karl Fischer assay.

The results indicate that the procedures provided in Examples 1 and 2 each succeeded in forming a crystalline salt comprising equimolar ratios of the acid and the pharmaceutical compound.

### EXAMPLE 4

This example presents an investigation of the dissolution rate characteristics of the sumanirole xinafoate crystalline salt of Example 2.

Dissolution rates of sumanirole from xinafoate and maleate crystalline salts were determined using a rotating disk apparatus. In this apparatus, a die containing a pellet comprising a substance of interest is attached to an arm connected to a small motor. The die is submerged in about 500 ml of a dissolution medium at 25° C., and rotated at 300 rpm. The concentration of a compound released into solution is measured in a series of time points using an in situ UV/vis absorbance dip probe linked to a digital computer. For testing the dissolution rates of sumanirole xinafoate and sumanirole maleate crystalline salts, 20 mg of the xinafoate crystalline salt and 20 mg of the maleate crystalline salt were each pressed into pellets of uniform shape at 1000 pounds (3700 psi) for 1 minute. Each pellet was then subjected to dissolution in a rotating disc dissolution apparatus. Measurements of the amount of sumanirole dissolved into the medium, recorded as milligrams of free base equivalent of sumanirole, were plotted against time (FIG. 1). The slope of each curve, normalized to the initial exposed surface area of each disk, was used to calculate a Rotating Disk Dissolution Rate (RDDR) for each salt. As shown in FIG. 1, the dissolution rate of the sumanirole xinafoate crystalline salt (RDDR=0.030 mmole sec⁻¹ cm⁻²) was less than 10% of the rate of the sumanirole maleate crystalline salt (RDDR=0.44 mmole sec⁻¹ cm⁻²).

As various changes could be made in the above methods and compositions without departing from the scope of the invention, it is intended that all matter contained in the above description be interpreted as illustrative and not in a limiting sense.

All references cited in this specification are hereby incorporated by reference in their entirety. The discussion of the references herein is intended merely to summarize the assertions made by their authors and no admission is made that any reference constitutes prior art relevant to patentability. Applicant reserves the right to challenge the accuracy and pertinency of the cited references.

What is claimed is:

1. A pharmaceutical composition comprising a salt, the salt comprising an organic acid having low water solubility and a compound of formula (I) 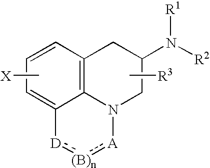
where
R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁ R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where [symbol] is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S, C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N.

2. A composition according to claim 1, wherein the organic acid has a water solubility of about 0.1% (w/w) or less.

3. A composition according to claim 1, wherein:
the organic acid is a compound selected from the group consisting of CH₃(CH₂)ₙCOOH, HOOC(CH₂)ₘCOOH, 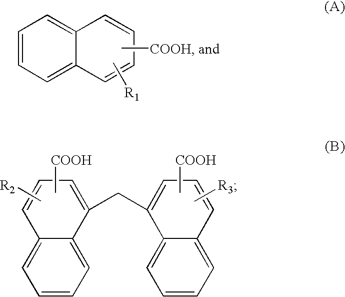
R¹, R₂, and R₃ are independently selected from the group consisting of hydroxy, amino, and nitro;
n is an integer from 5 to 16; and
m is an integer from 5 to 16.

4. A composition according to claim 3, wherein the organic acid is pamoic acid.

5. A composition according to claim 3, wherein the organic acid is xinafoic acid.

6. A composition according to claim 1, wherein the compound is selected from the group consisting of sumanirole, (R)-5,6-dihydro-5-(methylamino)-4H-imidazo[4,5-ij]-quinoline-2(1H)-thione and (R)-5,6-dihydro-N,N-dimethyl-4H-imidazo[4,5,1-ij]quinolin-5-amine.

7. A composition according to claim 5, wherein the compound is sumanirole.

8. A composition according to claim 1, wherein the salt has a dissolution rate into aqueous solution of about 0.40 mmole sec⁻¹ cm⁻² or less.

9. A composition according to claim 8, wherein the salt has a dissolution rate into aqueous solution of about 0.05 mmole sec⁻¹ cm⁻² or less.

10. A salt comprising an organic acid having low water solubility and a compound of formula (I) 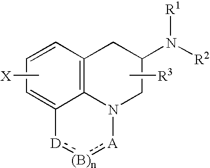
where
R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, NR₁R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where [symbol] is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and —A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S, C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N.

11. A salt according to claim 10, wherein the organic acid has a water solubility of about 0.1% (w/w) or less.

12. A salt according to claim 10, wherein:
the organic acid is a compound selected from the group consisting of CH₃(CH₂)ₙCOOH, HOOC(CH₂)ₘCOOH, 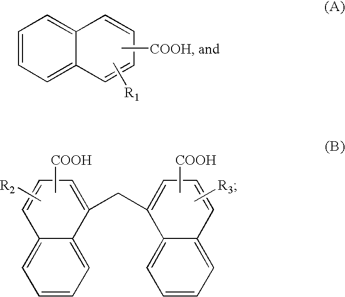
R₁, R₂, and R₃ are independently selected from the group consisting of hydroxy, amino, and nitro;
n is an integer from 5 to 16; and
m is an integer from 5 to 16.

13. A salt according to claim 12, wherein the organic acid is pamoic acid.

14. A salt according to claim 12, wherein the organic acid is xinafoic acid.

15. A salt according to claim 10, wherein the compound is selected from the group consisting of sumanirole, (R)-5,6-dihydro-5-(methylamino)-4H-imidazo[4,5-ij]-quinoline-2(1H)-thione and (R)-5,6-dihydro-N,N-dimethyl-4H-imidazo[4,5,1-ij]quinolin-5-amine.

16. A salt according to claim 15, wherein the compound is sumanirole.

17. A salt according to claim 10, wherein the salt has a dissolution rate into aqueous solution of about 0.40 mmole sec⁻¹ cm⁻² or less.

18. A salt according to claim 17, wherein the salt has a dissolution rate into aqueous solution of about 0.05 mmole sec⁻¹ cm⁻² or less.

19. A crystalline salt comprising an organic acid having low water solubility and a compound of formula (I) 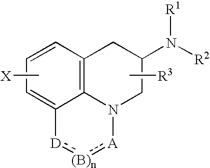
where
R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where [symbol] is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, ONH, N—CH₃;
(2) that when n is 0, and
A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S, C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N.

20. A crystalline salt according to claim 19, wherein the organic acid has a water solubility of about 0.1% (w/w) or less.

21. A crystalline salt according to claim 19, wherein:
the organic acid is a compound selected from the group consisting of CH₃(CH₂)ₙCOOH, HOOC(CH₂)ₘCOOH, 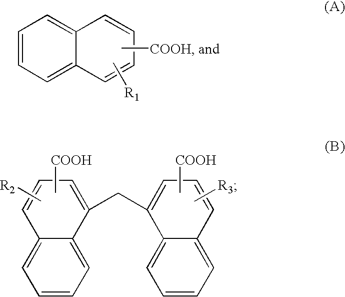
R₁, R₂, and R₃ are independently selected from the group consisting of hydroxy, amino, and nitro;
n is an integer from 5 to 16; and
m is an integer from 5 to 16.

22. A crystalline salt according to claim 21, wherein the organic acid is pamoic acid.

23. A crystalline salt according to claim 21, wherein the organic acid is xinafoic acid.

24. A crystalline salt according to claim 19, wherein the compound is selected from the group consisting of sumanirole, (R)-5,6-dihydro-5-(methylamino)-4H-imidazo[4,5-ij]-quinoline-2(1H)-thione and (R)-5,6-dihydro-N,N-dimethyl-4H-imidazo[4,5,1-ij]quinolin-5-amine.

25. A crystalline salt according to claim 24, wherein the compound is sumanirole.

26. A crystalline salt according to claim 19, wherein the salt has a dissolution rate into aqueous solution of about 0.40 mmole sec⁻¹ cm⁻² or less.

27. A crystalline salt according to claim 26, wherein the salt has a dissolution rate into aqueous solution of about 0.05 mmole sec⁻¹ cm⁻² or less.

28. An extended release composition comprising a salt of an organic acid having low water solubility and a compound of formula (I) 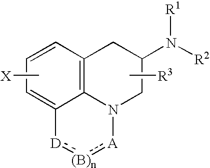
and a pharmaceutically acceptable polymer which modulates the rate of release of the compound into an aqueous phase,
wherein
R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where [symbol] is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S, C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N.

29. An extended release composition according to claim 28, wherein the organic acid has a water solubility of about 0.1% (w/w) or less.

30. An extended release composition according to claim 28, wherein:
the organic acid is a compound selected from the group consisting of CH₃(CH₂)ₙCOOH, HOOC(CH₂)ₘCOOH, 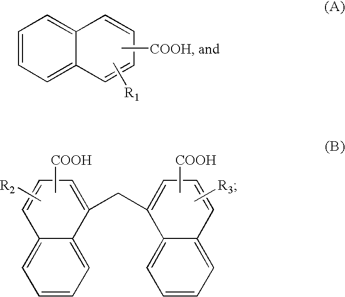
R₁, R₂, and R₃ are independently selected from the group consisting of hydroxy, amino, and nitro;
n is an integer from 5 to 16; and
m is an integer from 5 to 16.

31. An extended release composition according to claim 30, wherein the organic acid is pamoic acid.

32. An extended release composition according to claim 30, wherein the organic acid is xinafoic acid.

33. An extended release composition according to claim 28, wherein the compound is selected from the group consisting of sumanirole, (R)-5,6-dihydro-5-(methylamino)-4H-imidazo[4,5-ij]-quinoline-2(1H)-thione and (R)-5,6-dihydro-N,N-dimethyl-4H-imidazo[4,5,1-ij]quinolin-5-amine.

34. An extended release composition according to claim 33, wherein the compound is sumanirole.

35. An extended release composition according to claim 28, wherein the salt has a dissolution rate into aqueous solution of about 0.40 mmole sec⁻¹ cm⁻² or less.

36. An extended release composition according to claim 35, wherein the salt has a dissolution rate into aqueous solution of about 0.05 mmole sec⁻¹ cm⁻² or less.

37. An extended release composition according to claim 28 wherein the pharmaceutically acceptable polymer is a protein, a starch, a cellulose, or a synthetic polymer; wherein said protein is a bovine, porcine, or ovine gelatin; wherein said starch is a corn or wheat starch; which is a pregelatinized starch or in the form of a sodium starch glycolate; wherein said cellulose is hydroxypropyl methylcellulose, a cellulose ether, or cellulose acetate; and wherein said synthetic polymer is a polyvinyl chloride, a polyvinyl acetate, a polyurethane, or a polyalkyl methacrylate.

38. An extended release composition according to claim 37 wherein the hydroxypropyl methylcellulose is selected from the group consisting of hydroxypropyl methylcellulose 2208 USP 100 cps, hydroxypropyl methylcellulose 2208 USP 4,000 cps, hydroxypropyl methylcellulose 2208 USP 15,000 cps, hydroxypropyl methylcellulose 2208 USP 100,000 cps, hydroxypropyl methylcellulose 2910 USP 4,000 cps, hydroxypropyl methylcellulose 2910 USP 10,000 cps, and mixtures thereof.

39. An extended release composition of claim 28 which is suitable for administration twice a day.

40. An extended release composition of claim 28 which is suitable for administration once a day.

41. A method for producing a crystalline salt, the crystalline salt comprising:
a compound of structure 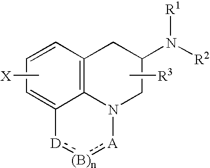
where R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where [symbol] is a single or double bond, with the provisos:
(1) that when is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S, C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N (formula (I)); and
an organic acid of structure 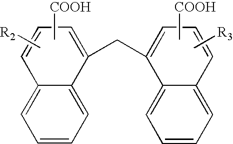
where R₂ and R₃ are independently selected from the group consisting of hydroxy, amino, and nitro (formula (B)), the method comprising:
(a) forming a mixture comprising the compound of structure formula (I), an organic acid of formula (B), and pyridine;
(b) evaporating the pyridine to form a solid;
(c) redissolving the solid in a solvent mixture comprising methanol and acetonitrile; and
(d) evaporating the solvent mixture, thereby precipitating the crystalline salt.

42. The method of claim 41 wherein the compound of formula (1) is selected from the group consisting of sumanirole, (R)-5,6-dihydro-5-(methylamino)-4H-imidazo[4,5-ij]-quinoline-2(1H)-thione, and (R)-5,6-dihydro-N,N-dimethyl-4H-imidazo[4,5,1 -ij]quinolin-5-amine.

43. The method of claim 41 wherein the compound of formula (I) is sumanirole.

44. The method of claim 41 wherein the organic acid of formula (B) is pamoic acid.

45. A method for producing a crystalline salt, the crystalline salt comprising:
a compound of structure 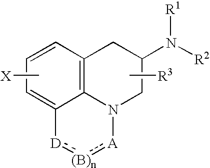
where R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where [symbol] is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S, C═NH, SO₂; and
B is CH₂, CH-(halogen) where halogen is as defined above, C═O, NH, N—CH₃; then
D is CH₂, C═O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N (formula (I)); and
an organic acid of structure 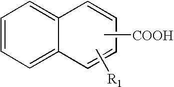
where R₁ is selected from the group consisting of hydroxy, amino, and nitro (formula (A)), the method comprising:
(a) forming a mixture of a compound of formula (I), the organic acid of formula (A), and methanol;
(b) evaporating the methanol to form an oil;
(c) redissolving the oil in acetonitrile; and
(d) evaporating the acetonitrile, thereby precipitating the crystalline salt.

46. The method of claim 45 wherein the compound of formula (I) is selected from the group consisting of sumanirole, (R)-5,6-dihydro-5-(methylamino)-4H-imidazo[4,5-ij]-quinoline-2(1H)-thione, and (R)-5,6-dihydro-N,N-dimethyl-4H-imidazo[4,5,1-ij]quinolin-5-amine.

47. The method of claim 45 wherein the compound of formula (I) is sumanirole.

48. The method of claim 45 wherein the organic acid of formula (A) is xinafoic acid.

49. A method of treating Parkinson's disease in a mammal in need thereof, the method comprising administering to the mammal a therapeutically effective amount of a composition comprising a salt, the salt comprising:
an organic acid having low water solubility; and
a compound of formula (I) 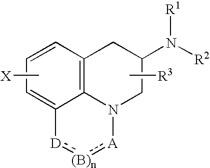
where
R¹, R² and R³ are the same or different and are: —H, C₁-C₆ alkyl, C₃-C₅ alkenyl, C₃-C₅ alkynyl, C₃-C₅ cycloalkyl, C₄-C₁₀ cycloalkyl, phenyl substituted C₁-C₆ alkyl, —NR₁R₂ where R₁ and R₂ are cyclized with the attached nitrogen atom to produce pyrrolidiyl, piperidinyl, morphoninyl, 4-methyl piperazinyl or imidazolyl;
X is: —H, C₁-C₆ alkyl, —F, —Cl, —Br, —I, —OH, C₁-C₆ alkoxy, cyano, carboxamide, carboxyl, (C₁-C₆ alkoxy)carbonyl;
A is: CH, CH₂, CH-(halogen) (where halogen is Cl, F, Br, or I), CHCH₃, C═O, C═S, C—SCH₃, C═NH, C—NH₂, C—NHCH₃, C—NHCOOCH₃, C—NHCN, SO₂, N;
B is: CH₂, CH, CH-(halogen) where halogen is as defined above, C═O, N, NH, N—CH₃,
D is: CH, CH₂, CH-(halogen) where halogen is as defined above, C═O, O, N, NH, N—CH₃; and n is 0 or 1, and where [symbol] is a single or double bond, with the provisos:
(1) that when n is 0, and
A is CH₂, CH-(halogen) where halogen is as defined above, CHCH₃, C═O, C═S, C═NH, SO₂; then
D is CH₂, CH-(halogen) where halogen is as defined above, C═O, O, NH, N—CH₃;
(2) that when n is 0, and
A is CH, C—SCH₃, C—NH₂, C—NHCH₃, C—NHCOOH₃, C—NHCN, N; then D is CH, N;
(3) that when n is 1, and
A is CH₂, CH-(halogen) where halogen is as defined above, CHCH₃, C═O, C—S, C═NH, SO₂; and
B is CH₂, CH-(halogen) where halogen is as defined above, C═O, NH, N—CH₃; then
D is CH₂, C═O, O, NH, N—CH₃;
(4) that when n is 1, and
A is CH, C—SCH₃, C—NH₂, C—NHCH₃, C—NHCOOCH₃, C—NHCN, N; and
B is CH, N; then
D is CH₂, C═O, O, NH, N—CH₃;
(5) that when n is 1, and
A is CH₂, CHCH₃, C═O, C═S, C═NH, SO₂, and
B is CH, N; then
D is CH, N.

50. A method according to claim 49, wherein the organic acid has a water solubility of about 0.1% (w/w) or less.

51. A method according to claim 50, wherein:
the organic acid is a compound selected from the group consisting of CH₃(CH₂)ₙCOOH, HOOC(CH₂)ₘCOOH, 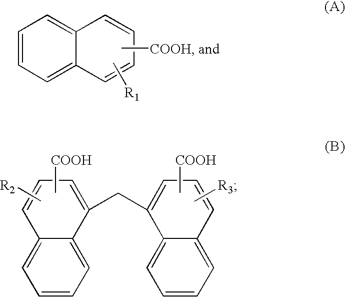
R₁, R₂, and R₃ are independently selected from the group consisting of hydroxy, amino, and nitro;
n is an integer from 5 to 16; and
n is an integer from 5 to 16.

52. A method according to claim 51, wherein the organic acid is selected from the group consisting of pamoic acid and xinafoic acid.

53. A method according to claim 49, wherein the compound is selected from the group consisting of sumanirole, (R)-5,6-dihydro-5-(methylamino)-4H-imidazo[4,5-ij]-quinoline-2(1H)-thione and (R)-5,6-dihydro-N,N-dimethyl-4H-imidazo[4,5,1-ij-]quinolin-5-amine.

54. A method according to claim 53, wherein the compound is sumanirole.

55. A method according to claim 49, wherein the salt has a dissolution rate into aqueous solution of about 0.40 mmole sec⁻¹ cm⁻² or less.

56. A method according to claim 55, wherein the salt has a dissolution rate into aqueous solution of about 0.05 mmole sec⁻¹ cm⁻² or less.

57. A method according to claim 49, wherein the administering is orally administering.

58. A method according to claim 49, wherein the mammal is a human.

59. A method according to claim 58, wherein the administering is no more often than about twice daily.

60. A method according to claim 58, wherein the administering is no more often than about once daily.

61. A method according to claim 49, wherein the therapeutically effective amount is from about 2 mg per day to about 120 mg per day.

62. A method according to claim 61, wherein the therapeutically effective amount is from about 3 mg per day to about 70 mg per day.
